(19) United States
(12) Patent Application Publication
(10) Pub. No.: US 2025/0062009 A1
(43) Pub. Date: Feb. 20, 2025

(54) ADAPTIVE NEUROMODULATOR ACTION POLICY GENERATION

(71) Applicant: International Business Machines Corporation, Armonk, NY (US)

(72) Inventors: Tigran Tigran Tchrakian, Castleknock (IE); Mykhaylo Zayats, Dublin (IE); Sergiy Zhuk, Dublin (IE); Djallel BOUNEFFOUF, Poughkeepsie, NY (US)

(73) Assignee: International Business Machines Corporation, Armonk, NY (US)

(21) Appl. No.: 18/233,725

(22) Filed: Aug. 14, 2023

(51) Int. Cl. G16H 20/70

(52) U.S. Cl. CPC G16H 20/70

(57) ABSTRACT

An embodiment collects a first set of patient data and a first set of treatment data associated with a patient population treated with neuromodulation. The embodiment clusters the patient population into a plurality of cohorts. The embodiment generates a plurality of states using a second set of patient data associated with a cohort in the plurality of cohorts. The embodiment generates a plurality of actions using a second set of treatment data associated with the cohort. The embodiment determines, based on the plurality of actions, a plurality of probabilities associated with a transition from a first state to a second state in the plurality of states. The embodiment generates, based on the plurality of probabilities, a neuromodulator action policy for a patient in the cohort.

### BACKGROUND

The present invention relates generally to neuromodulation. More particularly, the present invention relates to a method, system, and computer program for adaptive neuromodulator action policy generation.

Neuromodulation therapy refers to a type of medical treatment that involves the alteration or modulation of nerve activity through targeted delivery of a stimulus, such as electrical stimulation or chemical agents, to specific neurological sites in the body. The efficacy and effects of neuromodulation can be influenced by a myriad of parameters, both from the technological side and the biological side. On the technological end, factors like the fractionation of charge on an electrode and the specifics of the waveform parameters may play a role. These waveform parameters include the shape, frequency, amplitude, and duration of the electrical pulses delivered to the target neural tissues. Additionally, the neuromodulation hardware and the surgical specifics, such as electrode placement and orientation, can play a role in the treatment's efficacy. From a biological standpoint, individual patient physiology may play a role in determining the outcomes of the neuromodulation. Variabilities in a patient's body, from the exact neural tissue structures to the inherent electrical properties of the patient's neural system, can greatly influence the therapy's results.

Deep Brain Stimulation (DBS) is an illustrative example of such a neuromodulation technique. It involves implanting electrodes deep within specific areas of the brain and delivering electrical impulses to modulate abnormal or pathologic neural activity. The exact positioning of these electrodes, even on a millimeter-by-millimeter basis, can affect the outcome of the treatment. Therefore, two patients who might seem to be receiving identical DBS treatments based on their device's settings might, in reality, be receiving quite different therapies due to differences in electrode placements or individual neuroanatomy. Recognizing the unique interplay between individual physiology and technological parameters, practitioners often view neuromodulation therapies as highly personalized.

### SUMMARY

The illustrative embodiments provide for adaptive neuromodulator action policy generation.

An embodiment includes collecting a first set of patient data and a first set of treatment data associated with a patient population treated with neuromodulation. This step may involve gathering and compiling relevant information about patients who have undergone neuromodulation therapy. This comprehensive dataset forms the backbone of subsequent steps, paving the way for thorough data analysis and the formulation of personalized treatment strategies.

The embodiment also includes clustering the patient population into a plurality of cohorts. This step may involve categorizing patients into distinct groups or “cohorts” based on certain shared characteristics. Such categorization enables more targeted and efficient data analysis, as it allows the system to treat each cohort as a separate, relatively homogenous entity.

The embodiment also includes generating a plurality of states using a second set of patient data associated with a cohort in the plurality of cohorts. This step may involve taking a fresh set of patient data pertaining to a specific cohort and transforming it into a set of distinct “states.” Each state may represent a unique combination of patient characteristics and medical outcomes.

The embodiment also includes generating a plurality of actions using a second set of treatment data associated with the cohort. This step may involve interpreting a second set of treatment data corresponding to the same cohort, and generating a set of “actions” from this data. Each action may correspond to a specific type of treatment, treatment strategy, or treatment outcome associated with neuromodulation.

The embodiment also includes determining a plurality of probabilities associated with a transition from a first state to a second state in the plurality of states. This step may involve estimating the likelihood of transitions between different states, based on the patterns observed in the data. These probabilities may provide a probabilistic model of how patient outcomes can evolve over time in response to different treatment actions.

The embodiment also includes generating a neuromodulator action policy for a patient in the cohort based on the plurality of probabilities. This step may involve using the calculated probabilities to derive a recommended course of action, or “action policy,” for the neuromodulator in treating a patient from the cohort. This policy may offer specific, data-driven guidance on how to adjust the neuromodulator to achieve optimal treatment outcomes.

The whole process constitutes an innovative, data-driven approach to optimizing the use of neuromodulation. By gathering comprehensive data, clustering patients into cohorts, and using these cohorts to generate states, actions, and transition probabilities, the system can derive personalized action policies for neuromodulation therapy. This approach is technically advantageous as it offers potential improvements in the effectiveness, efficiency, and personalization of a neuromodulation treatment, such as DBS treatment.

An embodiment may include determining a current state of the patient using time-series patient data associated with the patient and determining the neuromodulator action policy by selecting one or more actions with the highest probability to transition from the current state to another state. This approach may allow for dynamic adjustment of the action policy based on the most current patient data, enabling more responsive and potentially more effective treatment strategies.

An embodiment may also include determining a current setting of a neuromodulator using time-series treatment data associated with the patient, and determining the neuromodulator action policy by selecting a sequence of adjustments to the neuromodulator with the highest probability to transition from the current state to another state. This approach may ensure that the action policy takes into account both the current state of the patient and the current settings of the neuromodulator, potentially optimizing the treatment outcome.

An embodiment may also include generating a Markov decision process using time-series patient data and time-series treatment data associated with the cohort, and determining the plurality of probabilities using the Markov decision process. This approach employs a mathematical framework for decision-making in uncertain environments, allowing for robust, probabilistic predictions of treatment outcomes.

An embodiment may also include receiving user feedback on the neuromodulator action policy and adjusting the Markov decision process based on the user feedback. This feature may allow the system to learn from user feedback, adapting its decision-making processes over time to potentially improve future treatment outcomes.

An embodiment also includes where a cohort represents a group of patients who share a similarity in at least one of a physiological feature, a symptom diagnosis, a neuromodulator device type, and an implant location. This categorization may help ensure that the system's analyses and recommendations are relevant to the specific characteristics and needs of each cohort.

An embodiment may also include presenting the neuromodulator action policy to the patient, and automatically adjusting a neuromodulator setting based on the neuromodulator action policy. This feature may combine transparent communication of the recommended treatment strategy with automated implementation of this strategy, enhancing both patient understanding and treatment efficiency.

An embodiment may also include where the patient data includes at least one of a symptom level, an activity level, a sleep quality, and a mood level; and a state in the plurality of states includes a combination of at least one of the symptom level, the activity level, the sleep quality, and the mood level. This approach may help ensure that the system considers a broad range of potentially relevant patient data in formulating its treatment recommendations, and allows for detailed, multi-dimensional modeling of patient states.

An embodiment may include where the treatment data includes at least one of a neuromodulator frequency, a neuromodulator current, a neuromodulator voltage, and a neuromodulator intensity; and the neuromodulator action policy includes adjusting at least one of the neuromodulator frequency, the neuromodulator current, the neuromodulator voltage, and the neuromodulator intensity. This specification may help ensure that the system can manipulate a broad range of neuromodulator settings in its quest to optimize treatment outcomes, and also makes it clear that the recommended action policies will involve specific, actionable adjustments to these settings.

A combination includes generating a Markov decision process using time-series patient data and time-series treatment data, determining probabilities using this process, receiving user feedback on the neuromodulator action policy, and adjusting the Markov decision process based on this feedback. This embodiment involves adjusting various neuromodulator settings, such as the frequency, current, voltage, and intensity. This combination provides the technical advantage of integrating user feedback into the decision-making process, potentially leading to improved patient satisfaction and compliance with the treatment plan. Furthermore, it leverages the power of the Markov decision process to make robust, probabilistic predictions about treatment outcomes, thereby optimizing the neuromodulator settings.

A specific implementation of this system could be in the management of symptom levels, such as tremors due to Parkinson's during DBS treatment. In this context, the system could be applied to continuously monitor and manage a Parkinson's patient's tremor levels using an implantable DBS device. The system would collect time-series data on the patient's tremor severity, activity level, sleep quality, and mood, as well as data on the current settings of the DBS device. The system would then utilize this data to create a Markov decision process, which would guide decisions on adjusting the DBS device settings. Throughout the treatment period, feedback from the patient regarding the impact of the DBS adjustments on their tremors would be constantly assimilated. This feedback would further refine the Markov decision process, resulting in an ever-evolving and more precise action policy. The ultimate goal being enhanced tremor management for the individual.

An embodiment includes a computer usable program product. The computer usable program product includes a computer-readable storage medium, and program instructions stored on the storage medium.

An embodiment includes a computer system. The computer system includes a processor, a computer-readable memory, and a computer-readable storage medium, and program instructions stored on the storage medium for execution by the processor via the memory.

### BRIEF DESCRIPTION OF THE DRAWINGS

The novel features believed characteristic of the invention are set forth in the appended claims. The invention itself, however, as well as a preferred mode of use, further objectives, and advantages thereof, will best be understood by reference to the following detailed description of the illustrative embodiments when read in conjunction with the accompanying drawings, wherein:

FIG. 1 depicts a block diagram of a computing environment in accordance with an illustrative embodiment.

FIG. 2 depicts a block diagram of an example software integration process in accordance with an illustrative embodiment.

FIG. 3 depicts a block diagram of an example neuromodulator action policy generator in accordance with an illustrative embodiment.

FIG. 4 depicts a block diagram of an example process for cohort generation in accordance with an illustrative embodiment.

FIG. 5 depicts a block diagram of an example process for neuromodulator setting clustering in accordance with an illustrative embodiment.

FIG. 6 depicts a block diagram of an example process for generating a neuromodulator action policy in accordance with an illustrative embodiment.

FIG. 7 depicts a block diagram of an example process for adaptive neuromodulator action policy generation in accordance with an illustrative embodiment.

### DETAILED DESCRIPTION

Neuromodulation therapy represents an approach in medical treatments that directly targets and modulates nerve activity. This is achieved by delivering specific stimuli, which can be electrical, chemical, or magnetic, to predetermined neurological sites within the body. Such therapies have been employed to manage a range of medical conditions, from chronic pain to movement disorders. Disorders that are often treated with neuromodulation therapies include, but are not limited to, Parkinson's disease, diabetic neuropathy, dystonia, epilepsy, essential tremor, hearing loss, Tourette's disorder, depression, and chronic pain. The intricacy of neuromodulation lies in its dependency on a multitude of parameters. Technologically, design specifics such as the fractionation of charge on the electrode or the waveform parameters, which determine the shape, frequency, amplitude, and duration of the electrical pulses, may play a role in the therapeutic outcome.

From a biological perspective, moreover, the efficacy of neuromodulation is intertwined with the unique physiological characteristics of each patient. For example, an individual's neural tissue structures, inherent electrical properties of their neural system, and their metabolism can influence the results of the therapy. Such intrinsic biological variations mean that even slight differences can lead to substantial deviations in therapeutic outcomes. Therefore, alongside the technological aspects, the patient's individual physiology may shape the therapy's impact. Moreover, the physical details of the neuromodulation hardware, as well as surgical specifics like electrode placement and orientation, may influence the therapy's success.

Deep Brain Stimulation (DBS) exemplifies the intricate nature of neuromodulation therapies. DBS involves surgically implanting electrodes within specific brain areas, targeting them with electrical impulses to regulate or alter abnormal neural activities. A slight deviation in electrode placement, even by a few millimeters, can render different therapeutic outcomes. Thus, two patients with seemingly identical DBS settings might experience vastly different results, all because of minute differences in the position of the electrodes or their unique brain structures. Given these nuances, neuromodulation therapies are often perceived as highly personalized treatments.

A significant challenge with prevailing methods for administrating neuromodulation therapies, such as DBS, is the absence of a streamlined strategy for tailoring treatments to individual needs. Conventional protocols largely hinge on an empirical approach where healthcare professionals tweak the parameters of the neuromodulation device in response to the feedback received from the patient. While this method might eventually pinpoint an effective therapeutic regimen, it is frequently an extended, inefficient journey marked by discomfort and patient dissatisfaction. This methodology also necessitates numerous clinical visits, posing inconvenience for patients and adding strain to the healthcare system.

A further complication arises from the intricate task of accurately recognizing and integrating all the variables influencing a patient's reaction to neuromodulation therapy. These variables may encompass not just the technological components of the intervention, such as electrode placement and device configurations, but also extend to unique patient-specific facets like their physiological nuances, overall health metrics, and daily lifestyle habits. Current strategies for orchestrating neuromodulation treatments often overlook these multifaceted determinants, culminating in less-than-ideal therapeutic results. Furthermore, leaning heavily on patient self-reporting as a barometer for gauging therapeutic effectiveness introduces potential for discrepancies. Given the subjective nature of sensations like pain or discomfort, patients' interpretations can vary widely.

The present disclosure addresses the deficiencies described above by providing a process (as well as a system, method, machine-readable medium, etc.) that facilitates personalized treatment recommendations for patients undergoing neuromodulation therapies, such as DBS.

Illustrative embodiments provide for the collection of patient data and treatment data. “Patient data,” as used herein, may refer to any data describing a patient's condition. This data can be collected in various ways, such as through medical records, medical devices, wearable sensors, self-complete patient questionnaires, doctors' visits, and more. For example, patient data might include information on symptom levels (e.g., tremors for Parkinson's), physiological parameters like heart rate or blood pressure, and lifestyle factors like activity levels or sleep quality. In some embodiments, for instance, patient data may include at least one of a symptom level, an activity level, a sleep quality, and a mood level. “Treatment data,” as used herein, may refer to the details of the medical treatment that a patient receives. For instance, it can capture the DBS settings applied to the patient. Treatment data could encompass parameters of the DBS device such as pulse width, frequency, and amplitude, along with the location and orientation of the implanted leads. In some embodiments, for example, treatment data may include at least one of a neuromodulator frequency, a neuromodulator current, a neuromodulator voltage, and a neuromodulator intensity.

Patient data and treatment data may be gathered using a variety of tools and methodologies to gather information about a patient's state and treatment. For example, sensors could be used to collect physiological data such as heart rate or skin conductance, while questionnaires could capture information about a patient's symptom levels, activity levels, or quality of life. Other data collection approaches could involve the extraction of treatment data from the neuromodulator device itself, or the collection of medical history and diagnosis information from medical records.

Illustrative embodiments provide for collecting a first set of patient data and a first set of treatment data associated with a patient population treated with neuromodulation. This process may include the assimilation of both subjective data (like patient-reported symptoms or comfort levels) and objective data (like sensor readings from the DBS device or other physiological measurements). For example, the system could collect patient-reported symptom levels, activity metrics from wearable devices, and data regarding the frequency and intensity settings of the DBS device over a certain period. This process could involve using embedded sensors within DBS devices, wearable devices for activity tracking, or self-report mechanisms through user-friendly applications to collect a diverse array of patient and treatment data. For instance, data points such as heart rate variability, skin temperature, patient-reported symptom scores, neuromodulator settings, and the duration of DBS usage could be gathered over time.

Illustrative embodiments provide for generating a plurality of cohorts. A “cohort,” as used herein, may refer to a group of patients segmented based on specific shared characteristics. These can include the treatment parameters, such as hardware type and implantation location, as well as aspects of patient physiology, including relevant diagnoses. For example, one cohort might consist of patients with a specific type of DBS device implanted at a certain location, who may also share a common diagnosis like Parkinson's. Another cohort might include patients with a different type of device or a different implantation location, or patients with a different neurological diagnosis.

Illustrative embodiments provide for clustering the patient population into a plurality of cohorts. This process may involve using clustering and/or machine learning techniques to group patients based on similarities in their data profiles. For example, patients could be clustered into groups based on shared characteristics such as similar symptom levels, similar responses to specific neuromodulator settings, or similar physiological markers. This process may involve the use of machine learning techniques, such as k-means clustering or hierarchical clustering, to categorize patients based on the multidimensionality of their data. This process might involve vectorization of patients' physiological features, symptom diagnosis, neuromodulator device types, implant locations, or treatment responses, among others, and then clustering them into similar groups.

In some embodiments, a cohort may represent a group of patients who share a similarity in at least one of a physiological feature, a symptom diagnosis, a neuromodulator device type, and an implant location. This process may involve grouping patients based on shared characteristics to better understand patterns and tailor treatment. For example, a cohort could be composed of patients who all have the same type of DBS device, experience similar levels of symptoms, or have the same implant location. This step could involve the use of multivariate statistical techniques or principal component analysis (PCA) to identify key features that characterize the cohort, allowing the system to effectively stratify the patient population based on those defining features.

Illustrative embodiments provide for the discovery of a set of states characterizing a cohort. A “state,” as used herein, may refer to a distinct condition or circumstance that describes the patients in a cohort, based on the aggregated data of patients belonging to that cohort. For example, a state could describe a combination of symptom levels, physiological metrics, and lifestyle factors. Within one cohort, states might vary from “high symptom levels, low activity” to “low symptom levels, high activity.” In a different cohort, the states might be defined differently based on the unique characteristics and circumstances of its patients. In some embodiments, for example, a state may include a combination of a symptom level, an activity level, a sleep quality, and a mood level.

Illustrative embodiments provide for defining multiple states using a second set of patient data related to a cohort within the plurality of cohorts. This could involve analyzing patterns to identify distinct patient states within each cohort. For instance, the system could identify states like “severe symptoms, sedentary behavior, poor sleep” or “mild symptoms, active behavior, good sleep.” States might be identified based on patterns observed in the second set of patient data. Techniques such as unsupervised learning or hidden Markov models could be used to uncover these states within the data. For instance, each state might be characterized by a unique combination of factors like symptom levels, activity patterns, sleep quality, and emotional well-being.

Illustrative embodiments provide for identifying an action for a cohort. An “action,” as used herein, might refer to common neuromodulator settings or parameters used by patients within a cohort. For instance, within one cohort, an action might represent a specific setting of the neuromodulator device commonly used by its members. In another cohort, the actions might be different, reflecting alternative neuromodulator parameters tailored to the unique characteristics of that cohort.

Illustrative embodiments provide for generating a plurality of actions using a second set of treatment data associated with the cohort. This process may involve analyzing the treatment data to identify the different therapeutic actions taken over time and their corresponding effects on the patient's state. For instance, the system could determine actions such as adjusting the DBS stimulator's voltage, frequency, or intensity. This process might employ pattern recognition or data mining algorithms to the treatment data to discern correlations between specific neuromodulator settings (e.g., frequency, intensity, and waveform type) and subsequent changes in patient states. It could also entail understanding the effects of different stimulation parameters on the transitions between patient states.

Illustrative embodiments provide for generating probabilities of state transitions for each cohort. A “state transition,” as used herein, may reference a change in a patient's state over time, conditioned on each cohort's actions. For instance, the system might discern that for a certain cohort, a specific action has a high likelihood of prompting a transition from a state of “severe tremors, low activity” to “mild tremors, high activity.”

The generation of state transition probabilities might involve training a machine learning model with the accumulated data specific to each distinct cohort. This data might encompass variables such as physiological metrics like heart rate, blood pressure, and sleep patterns, patient-reported symptoms, mood variations, and responses to distinct neuromodulator settings. Additionally or alternatively, the machine learning model might be fed with a sequence of state transition probabilities coupled with their correlated actions, defined per cohort. By analyzing and learning from this data, the machine learning model can pinpoint patterns and forecast results. This insight into the influence of various treatment settings on patient states might empower the system to refine neuromodulation therapy, rendering it more personalized and efficacious.

Illustrative embodiments provide for determining, based on the plurality of actions, a range of probabilities linked with a transition from an initial state to a subsequent state in the assortment of states. This process might involve deducing the likelihood of transitioning from one patient state to another in light of specific therapeutic actions. For instance, the system could approximate the probability of a patient moving from a “severe tremors” state to a “mild tremors” state following a modification in the DBS stimulator's frequency. This procedure might integrate learning algorithms to approximate the transition probabilities between states according to the actions executed. Techniques like a Markov decision process or Q-learning algorithm could be utilized to construct a table signifying the anticipated rewards for all feasible state-action combinations.

In some embodiments, the learning process may entail producing and employing a Markov decision process (MDP), a mathematical framework adept at modeling decision-making situations where results are partly stochastic and partly under the discretion of a decision-maker. In this context, it might be utilized to grasp and navigate the states and actions tied to neuromodulation therapy. For instance, the MDP could assess the transition probabilities between diverse patient states given a variety of actions (like differing DBS settings), thereby enabling the system to predict and modify patient outcomes with enhanced accuracy and efficiency. The MDP can learn and model the likelihoods of transitioning from one patient state to the next, considering the effects of various DBS treatment settings. For example, the MDP might discover that a particular DBS setting augments the probability of a patient with Parkinson's transitioning from a “severe tremors” state to a “mild tremors” state.

Illustrative embodiments provide for generating a neuromodulator action policy. A “neuromodulator action policy.” as used herein, may refer to a set of recommendations that dictate the most favorable actions or neuromodulation device settings (e.g., a DBS device) for a patient, given their current state and the historical behavior of their cohort. In some embodiments, for example, the neuromodulator action policy may include adjusting at least one of a neuromodulator frequency, a neuromodulator current, a neuromodulator voltage, and a neuromodulator intensity. The central goal of this policy may be to facilitate a patient's transition to an optimal state over a specified period. The optimal state may represent the most desirable condition for a patient, which could be characterized by reduced symptoms and improved functionality. As an example, the system, utilizing a pre-trained MDP, might recommend a patient to adjust their DBS settings in a specific way, such as increasing the pulse width while decreasing the frequency. This recommendation is based on its learned understanding that this particular setting adjustment has a high likelihood of transitioning the patient to a state of reduced symptoms and improved functionality.

Illustrative embodiments provide for generating a neuromodulator action policy for a patient in the cohort. This process might involve leveraging the probabilities and identified states to design a policy dictating the optimal neuromodulator settings for a patient. For instance, if the system has learned that a specific action frequently leads to a positive state transition for similar patients within the same cohort, that action might be included in the policy for the patient. This process might involve the application of decision theory principles and optimization algorithms to recommend the most beneficial set of neuromodulator actions based on the calculated probabilities. For instance, it might suggest a specific neuromodulator frequency that, according to the learned probabilities, maximizes the likelihood of transitioning a patient to a more desirable state (e.g., reduced symptoms, improved sleep).

Illustrative embodiments provide for determining a current state of the patient using time-series patient data associated with the patient; and determining the neuromodulator action policy by selecting one or more actions with the highest probability to transition from the current state to another state. This process may involve using real-time data to assess the patient's current state, and then leveraging the previously developed policy to choose the action(s) most likely to facilitate a transition to a more desirable state. For example, if the patient's current state is “severe tremors, low mobility,” the system might recommend increasing the DBS's intensity, based on the learned knowledge that this action often leads to reduced tremors and improved mobility. This process might involve using recurrent neural networks (RNNs) or long short-term memory (LSTM) networks for processing time-series data to determine the patient's current state. The system could then select actions based on the Markov decision process or Q-table that maximize the expected future reward, leading to a beneficial state transition.

Illustrative embodiments provide for determining a current setting of a neuromodulator using time-series treatment data associated with the patient; and determining the neuromodulator action policy by selecting a sequence of adjustments to the neuromodulator device with the highest probability to transition from the current state to another state. This process might involve analyzing the past and present settings of the patient's neuromodulator device, and then using this data, in conjunction with the patient's current state, to identify a sequence of adjustments likely to lead to a state transition. For instance, if the patient's current state is “poor sleep, severe tremors,” the system could suggest a sequence of changes to the DBS's settings that, based on historic data, has shown to improve sleep and reduce tremors. This process could involve integrating time-series forecasting techniques with reinforcement learning to predict the optimal sequence of adjustments to the neuromodulator settings, thereby facilitating a successful transition from the current state to a more desirable state.

Illustrative embodiments provide for generating an MDP using time-series patient data and time-series treatment data associated with the cohort; and determining the plurality of probabilities using the MDP. This process may involve creating a dynamic, probabilistic model that captures the possible transitions between different patient states. The MDP may integrate information about both patient states and treatment actions, enabling the system to calculate transition probabilities and thereby devise optimal policies. The creation of the MDP could involve mapping patient states and actions onto a graph-like structure, with the edges representing the probabilities of transitions between states given certain actions. The probabilities could be determined by analyzing the frequency of state transitions in the historical data.

Illustrative embodiments provide for receiving user feedback on the neuromodulator action policy and/or adjusting the MDP based on the user feedback. This process may include an iterative loop wherein the patient or their healthcare provider offers feedback on the recommended neuromodulator action policy, and this feedback is used to refine the underlying model, thus improving future recommendations. For example, if a patient reports that a specific neuromodulator action did not yield the expected improvement, the system could adjust the MDP to reflect this feedback. This step could involve integrating user feedback mechanisms into the user interface and employing Bayesian updating techniques to adjust the transition probabilities in the MDP. For example, the system could use patient-reported outcomes to revise the probabilities associated with specific state-action pairs, thereby improving the accuracy and effectiveness of the system's recommendations over time.

Illustrative embodiments provide for presenting the neuromodulator action policy to the patient; and automatically adjusting a neuromodulator setting based on the neuromodulator action policy. This process might involve communicating the recommended changes in the neuromodulator settings to the patient via a user-friendly interface, or autonomously adjusting the neuromodulator settings. For instance, the system could display the recommended adjustments to the patient through a smartphone application, or it could directly modify the neuromodulator's settings without requiring manual input from the patient or their healthcare provider. This process could involve developing a user-friendly graphical user interface (GUI) that clearly communicates the recommended neuromodulator settings to the patient, or integrating the system with the neuromodulator's control unit to allow for automatic adjustments to the device's settings based on the system's recommendations.

For the sake of clarity of the description, and without implying any limitation thereto, the illustrative embodiments are described using some example configurations. From this disclosure, those of ordinary skill in the art will be able to conceive many alterations, adaptations, and modifications of a described configuration for achieving a described purpose, and the same are contemplated within the scope of the illustrative embodiments.

Furthermore, simplified diagrams of the data processing environments are used in the figures and the illustrative embodiments. In an actual computing environment, additional structures or components that are not shown or described herein, or structures or components different from those shown but for a similar function as described herein may be present without departing the scope of the illustrative embodiments.

Furthermore, the illustrative embodiments are described with respect to specific actual or hypothetical components only as examples. Any specific manifestations of these and other similar artifacts are not intended to be limiting to the invention. Any suitable manifestation of these and other similar artifacts can be selected within the scope of the illustrative embodiments.

The examples in this disclosure are used only for the clarity of the description and are not limiting to the illustrative embodiments. Any advantages listed herein are only examples and are not intended to be limiting to the illustrative embodiments. Additional or different advantages may be realized by specific illustrative embodiments. Furthermore, a particular illustrative embodiment may have some, all, or none of the advantages listed above.

Furthermore, the illustrative embodiments may be implemented with respect to any type of data, data source, or access to a data source over a data network. Any type of data storage device may provide the data to an embodiment of the invention, either locally at a data processing system or over a data network, within the scope of the invention. Where an embodiment is described using a mobile device, any type of data storage device suitable for use with the mobile device may provide the data to such embodiment, either locally at the mobile device or over a data network, within the scope of the illustrative embodiments.

The illustrative embodiments are described using specific code, computer readable storage media, high-level features, designs, architectures, protocols, layouts, schematics, and tools only as examples and are not limiting to the illustrative embodiments. Furthermore, the illustrative embodiments are described in some instances using particular software, tools, and data processing environments only as an example for the clarity of the description. The illustrative embodiments may be used in conjunction with other comparable or similarly purposed structures, systems, applications, or architectures. For example, other comparable mobile devices, structures, systems, applications, or architectures therefor, may be used in conjunction with such embodiment of the invention within the scope of the invention. An illustrative embodiment may be implemented in hardware, software, or a combination thereof.

The examples in this disclosure are used only for the clarity of the description and are not limiting to the illustrative embodiments. Additional data, operations, actions, tasks, activities, and manipulations will be conceivable from this disclosure and the same are contemplated within the scope of the illustrative embodiments.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation, or garbage collection, but this does not render the storage device as transitory because the data is not transitory while it is stored.

The process software for neuromodulator action policy generation is integrated into a client, server and network environment, by providing for the process software to coexist with applications, operating systems and network operating systems software and then installing the process software on the clients and servers in the environment where the process software will function.

The integration process identifies any software on the clients and servers, including the network operating system where the process software will be deployed, that are required by the process software or that work in conjunction with the process software. This includes software in the network operating system that enhances a basic operating system by adding networking features. The software applications and version numbers will be identified and compared to the list of software applications and version numbers that have been tested to work with the process software. Those software applications that are missing or that do not match the correct version will be updated with those having the correct version numbers. Program instructions that pass parameters from the process software to the software applications will be checked to ensure the parameter lists match the parameter lists required by the process software. Conversely, parameters passed by the software applications to the process software will be checked to ensure the parameters match the parameters required by the process software. The client and server operating systems, including the network operating systems, will be identified and compared to the list of operating systems, version numbers and network software that have been tested to work with the process software. Those operating systems, version numbers and network software that do not match the list of tested operating systems and version numbers will be updated on the clients and servers in order to reach the required level.

After ensuring that the software, where the process software is to be deployed, is at the correct version level that has been tested to work with the process software, the integration is completed by installing the process software on the clients and servers.

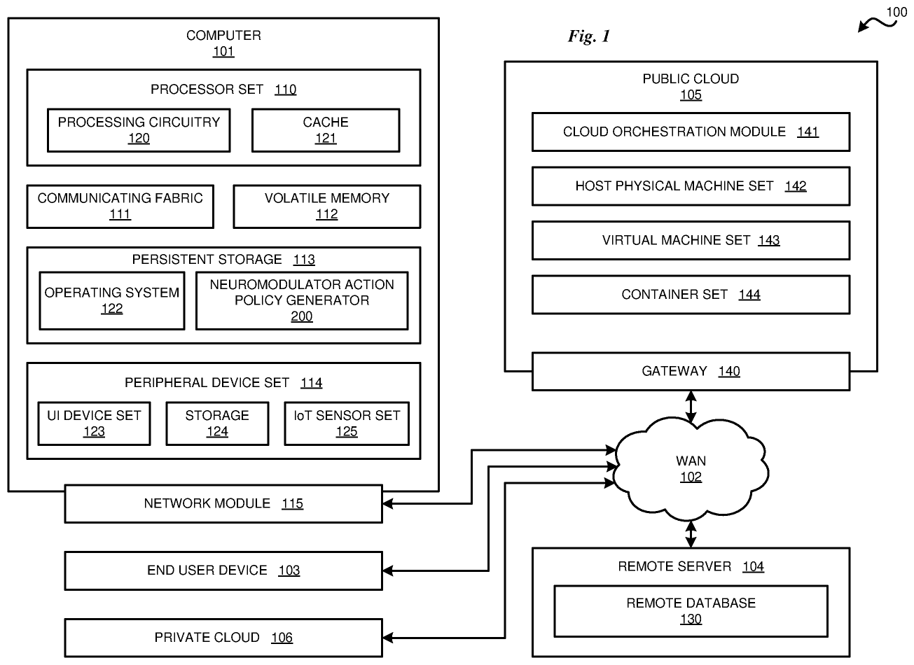

With reference to FIG. 1, this figure depicts a block diagram of a computing environment 100. Computing environment 100 contains an example of an environment for the execution of at least some of the computer code involved in performing the inventive methods, such as neuromodulator action policy generator 200 for adaptive neuromodulator action policy generation. In addition to block 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 200, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up buses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in block 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 012 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economics of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

Measured service: cloud systems automatically control and optimize resource use by leveraging a metering capability at some level of abstraction appropriate to the type of service (e.g., storage, processing, bandwidth, and active user accounts). Resource usage can be monitored, controlled, reported, and invoiced, providing transparency for both the provider and consumer of the utilized service.

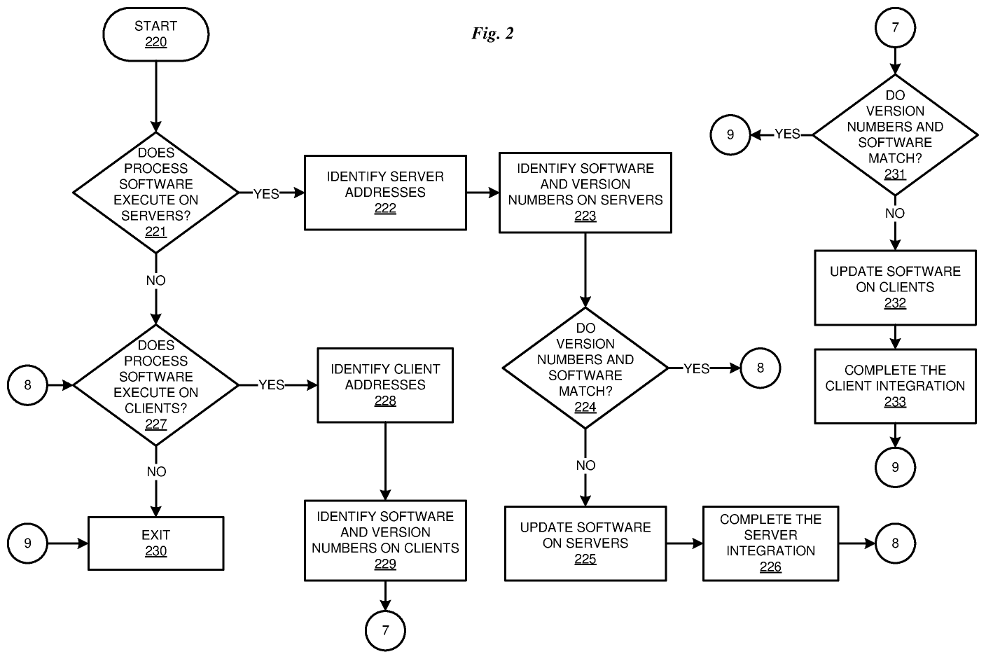

With reference to FIG. 2, this figure depicts a block diagram of an example software integration process, which various illustrative embodiments may implement. Step 220 begins the integration of the process software. An initial step is to determine if there are any process software programs that will execute on a server or servers (221). If this is not the case, then integration proceeds to 227. If this is the case, then the server addresses are identified (222). The servers are checked to see if they contain software that includes the operating system (OS), applications, and network operating systems (NOS), together with their version numbers that have been tested with the process software (223). The servers are also checked to determine if there is any missing software that is required by the process software (223).

A determination is made if the version numbers match the version numbers of OS, applications, and NOS that have been tested with the process software (224). If all of the versions match and there is no missing required software, the integration continues (227).

If one or more of the version numbers do not match, then the unmatched versions are updated on the server or servers with the correct versions (225). Additionally, if there is missing required software, then it is updated on the server or servers (225). The server integration is completed by installing the process software (226).

Step 227 (which follows 221, 224 or 226) determines if there are any programs of the process software that will execute on the clients. If no process software programs execute on the clients, the integration proceeds to 230 and exits. If this not the case, then the client addresses are identified (228).

The clients are checked to see if they contain software that includes the operating system (OS), applications, and network operating systems (NOS), together with their version numbers that have been tested with the process software (229). The clients are also checked to determine if there is any missing software that is required by the process software (229).

A determination is made if the version numbers match the version numbers of OS, applications, and NOS that have been tested with the process software (231). If all of the versions match and there is no missing required software, then the integration proceeds to 230 and exits.

If one or more of the version numbers do not match, then the unmatched versions are updated on the clients with the correct versions 232. In addition, if there is missing required software, then it is updated on the clients 232. The client integration is completed by installing the process software on the clients 233. The integration proceeds to 230 and exits.

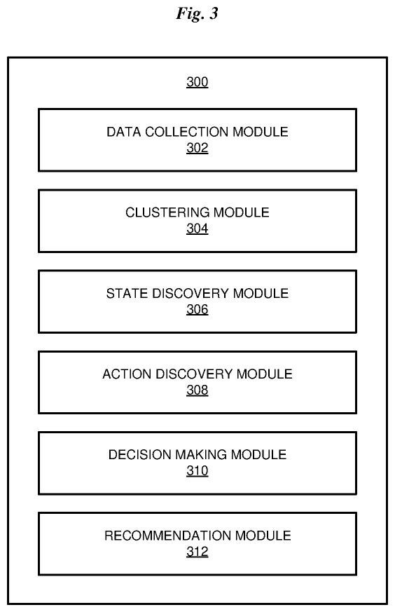

With reference to FIG. 3, this figure depicts a block diagram of an example neuromodulator action policy generator 300, which various illustrative embodiments may implement. It is to be understood that neuromodulator action policy generator 300 may be implemented as a single unit or as a part of a larger system in which computer-usable program code or instructions implementing the processes may be located for the illustrative embodiments. In addition, a neuromodulator action policy generator may comprise additional or fewer components than those shown in the illustrative embodiment.

In the depicted example, neuromodulator action policy generator 300 employs an architecture which includes a data collection module 302, a clustering module 304, a state discovery module 306, an action discovery module 308, a decision-making module 310, and a recommendation module 312.

Data collection module 302 may be configured to collect patient data and treatment data from a myriad of sources, including medical records and patient self-assessed questionnaires. These questionnaires may be tailored to capture a broad spectrum of patient-specific data points such as symptom level, mood, level of activity, medication intake volume, sleep duration, and more. The questionnaires may be digital or physical and may be designed to be easily understood and completed by patients. Moreover, the data collection module 302 may retrieve data from another device or system, such as neuromodulation information from a DBS device. This information may include technical parameters such as the number of leads, level of currents, voltage, intensity, the specific model of the device, and other pertinent data. The collected data from these sources may be cleaned, sorted, and prepared for further processing.

Clustering module 304 may be configured to process the data to group the patient population into one or more cohorts. This module may not only process the raw patient and treatment data collected by the previous module but also derive certain metrics from it. For instance, it may compute statistics like the average, minimum, and maximum values of given time series data such as symptom levels, mood fluctuations, device intensity, electrical currents, and others. The methodology behind the clustering can vary based on the dataset's characteristics. For instance, statistical methods such as K-means or density-based spatial clustering (DBSCAN) may be employed. The K-means method identifies “K” number of centroids, and each data point may be associated with the nearest centroid, thus forming “K” clusters or cohorts. DBSCAN is a machine learning algorithm that identifies clusters in a dataset based on the density of data points, distinguishing between high-density regions (clusters), lower density regions (noise), and transitional areas. Other methods for generating cohorts may be utilized, however, as would be appreciated by those having ordinary skill in the art upon reviewing the present disclosure.

State discovery module 306 may be configured to identify one or more states associated with the cohorts produced by the clustering module 304. For instance, it may take the aggregated physiological data from patients in each cohort and apply clustering techniques again. These could be similar to those used previously, such as K-means or DBSCAN, but adapted to the nature of the data. This module may construct clusters, and their centroids, referred to as “states,” which encapsulate the patient conditions most effectively. For instance, based on symptom levels, mood, and sleep quality data, the system might recognize a “best” state representing conditions like low symptom levels, good mood, and restful sleep, and a “worst” state, indicating high symptom levels, poor mood, and disrupted sleep.

Action discovery module 308 may be configured to identify one or more actions associated with the cohorts produced by the clustering module 304. For instance, it may be configured to work with the neuromodulation data specific to each cohort. Leveraging clustering methodologies akin to those used earlier, like K-means or DBSCAN, it may work on the neuromodulation data and form clusters. The resulting cluster centroids, termed “actions,” may illustrate the typical configuration of a neuromodulator (e.g., a DBS device) for that specific cohort. For example, a certain cohort might typically have a certain intensity and voltage setting, which may be captured as an action for that cohort.

Decision-making module 310 may be configured to learn the probabilities of state transitions for each cohort, conditioned on each of that cohort's actions. For instance, it may model these state transitions using a Markov decision process (MDP). An MDP is a mathematical framework used in decision making where outcomes are partly random and partly under the control of a decision-maker. The MDP may represent each patient's condition as a state and possible treatments as actions. The decision-making module may estimate the transition probabilities between states based on the action taken. For instance, it may calculate the likelihood of a patient moving from a “worst” state to a “best” state following a certain DBS device setting.

The training of the MDP may involve using historical patient data and actual outcomes to refine the probability estimates for different state transitions given specific actions. For example, if a patient, currently in a “worst” state defined by high symptom levels, poor mood, and disrupted sleep, applies a certain DBS device setting recommended by the system, and subsequently transitions into a “better” state characterized by improved mood, restful sleep, and decreased symptom levels, this information may be fed back into the MDP. The MDP may utilize this feedback to update its understanding of the likelihood of this particular state transition given the specific DBS setting. Over time, with the integration of more patient data and outcomes, the MDP may become increasingly proficient at predicting the potential results of various DBS settings on patient states. The MDP may be trained using machine learning algorithms, such as Q-learning or policy iteration, which allow it to continuously learn from new data and adapt its predictions accordingly.

Recommendation module 312 may be configured to provide a recommendation to guide a patient toward a favorable state. For instance, it may be configured to gather the most up-to-date information about a patient, determine the most suitable cohort for them, and recommend an optimal action from among the actions typically associated with that cohort. The module may perform this action by comparing the potential outcomes of different actions and recommending the one most likely to result in improved health outcomes. For example, it might suggest adjusting the intensity or voltage of a DBS device to improve symptom levels, mood, and sleep quality.

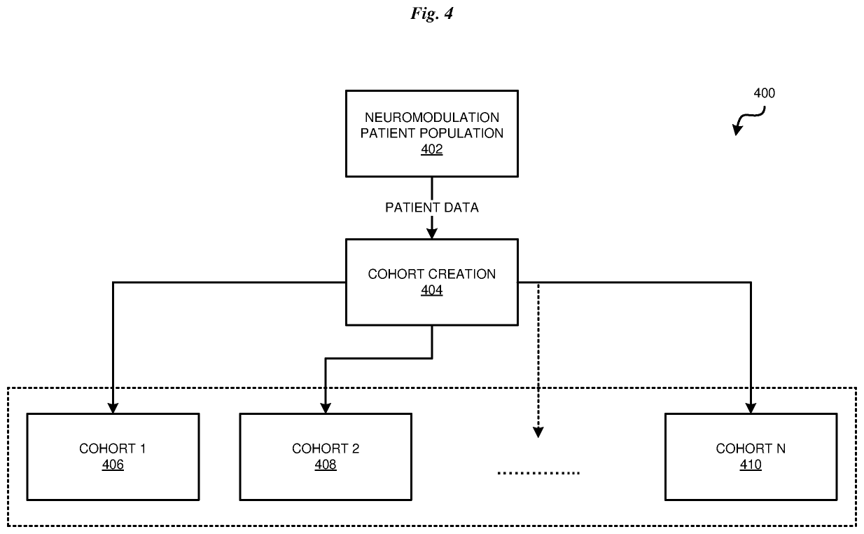

With reference to FIG. 4, this figure depicts a block diagram of an example process for cohort generation in accordance with an illustrative embodiment 400. The example block diagram of FIG. 4 may be implemented using neuromodulator action policy generator 200 of FIG. 1.

In the illustrative embodiment, the process may begin by accessing data related to a neuromodulation patient population 402. This patient population may represent a dataset of patients who are undergoing neuromodulation treatment. The data may encapsulate a broad spectrum of patient-related parameters such as physiological information (e.g., individual brain activity patterns, genetic predispositions), anatomical features (e.g., brain structure variations), symptom characteristics (type, location, severity), and treatment details (e.g., DBS device type, device settings). In addition to these, DBS device-specific parameters such as electrode charge configuration, waveform parameters, and lead-related data (types, locations) may be part of this dataset. Additionally, behavioral and subjective aspects like patient-reported symptom levels, mood, level of activity, medication usage, and sleep patterns, captured through self-assessed questionnaires or other patient engagement platforms, may be part of the dataset. Other data may be included, however, depending on the specific application.

This step may involve performing data retrieval and/or data integration. Data sources can vary from digital Electronic Health Records (EHR) systems to physical patient records, as well as databases maintained by neuromodulator device manufacturers that contain relevant device-specific information. Data retrieved from patient questionnaires—either paper-based or through digital health applications—may also be inputs. To ensure data quality, consistency checks, data validation, and cleansing methods may be applied. While integrating data from these diverse sources, data anonymization processes, in compliance with privacy laws such as GDPR or HIPAA, may be employed to uphold patient privacy.

After data collection, the process proceeds to block 404, where it may perform cohort generation. Cohort generation may involve the application of machine learning techniques to process and segment the collected data, resulting in distinct groups or cohorts. These cohorts may be formed based on shared characteristics among patients such as similarities in lead location or type, patient physiology, symptom profiles, or neuromodulator device types, among others. For example, a clustering algorithm might identify a group of patients who all share similar brain structures, experience Parkinson's symptoms, and are using a specific model of a DBS device with a particular electrode configuration. Machine learning clustering techniques that could be applied include K-means for partitioning, DBSCAN for density-based clustering, hierarchical clustering, spectral clustering, or clustering techniques based on deep learning models.

During this phase, the process may employ patient data from the neuromodulation patient population 402 to calculate metrics that aid the cohort generation process. This step may involve a time-series analysis of the collected data, through which derived metrics such as average, minimum, and maximum values of symptom levels, mood changes, device intensity, and electrical current usage may be computed. These metrics may provide a deeper understanding of the evolution of patients' conditions and responses to the neuromodulation treatment over time, thereby contributing nuanced details to the cohort generation process. Machine learning algorithms like Long Short-Term Memory (LSTM) or other recurrent neural network models could be used for time-series analysis, given their ability to handle temporal dependencies, although any other suitable process may be used.

As an outcome of this process, one or more cohorts may be generated, depicted as cohorts 406, 408, and 410. Each cohort may represent a set of characteristics and conditions within the neuromodulation patient population. They may be distinguished based on specific combinations of patient physiology, symptom profiles, neuromodulation device parameters, and/or treatment responses, among other information. Each of these cohorts may be visualized as a distinct cluster in a high-dimensional parameter space, where the dimensions may represent the variety of factors considered during clustering.

For instance, cohort 406 could represent a group of patients who share similar physiological characteristics, have Parkinson's symptoms, and are being treated with a particular model of DBS device. This cohort could further be characterized by other shared traits, like similar symptom thresholds or parallel symptom histories. For example, these patients might all respond positively to a certain range of device intensity settings. Additionally or alternatively, the shared trait could be a common behavioral pattern, such as consistently improved sleep quality post-treatment. To identify such commonalities, statistical analysis and pattern recognition techniques can be applied to the cohort's dataset.

Cohort 408 might represent a different patient population segment. For example, these patients might suffer from severe tremors that affect multiple parts of their bodies, thereby requiring a different treatment approach. They might be using a different model or type of neuromodulator device, designed specifically to address these symptoms. The shared characteristics among these patients could be the type of leads used in their DBS devices, which may be unique to addressing their specific symptom profile. Additionally or alternatively, commonality might be found in non-physiological traits like sleep patterns or daily activity levels. Sleep tracking technologies or activity monitors could be used to collect this data, and machine learning algorithms can help detect common patterns among the cohort members.

Lastly, cohort 410 may consist of patients dealing with complex neurological conditions who are undergoing neuromodulation therapy with advanced device settings. These patients might require more intensive neuromodulation, demonstrated by the high current and voltage levels in their neuromodulator devices. Moreover, their unique anatomical features might necessitate specific lead placements. Identifying shared characteristics within this cohort may require machine learning algorithms capable of discerning patterns within complex and multi-dimensional data. This step might include using neural networks (e.g., autoencoders for dimensionality reduction), and then applying clustering techniques to the reduced data for defining the cohort. Each of these cohorts can help in designing more personalized and effective neuromodulation programs, thereby improving the overall efficacy of the neuromodulation treatment.

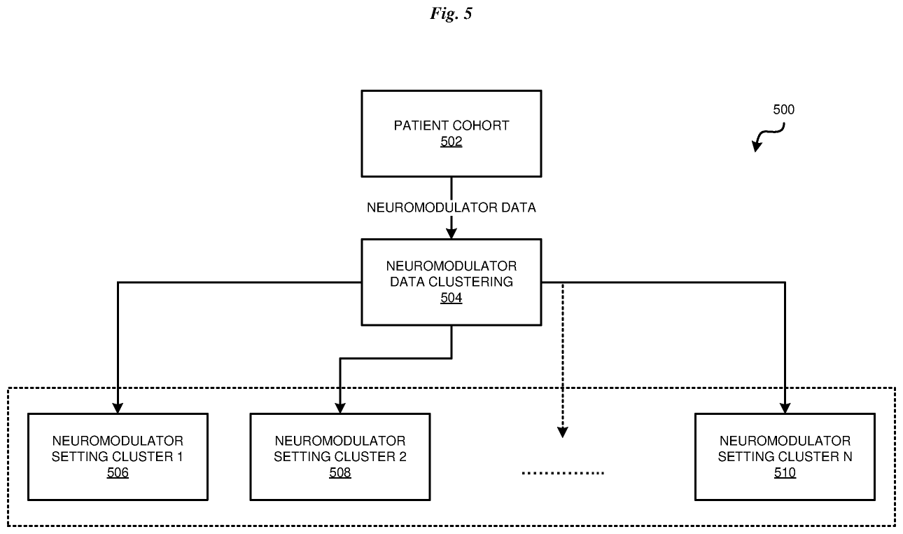

With reference to FIG. 5, this figure depicts a block diagram of an example process for neuromodulator setting clustering in accordance with an illustrative embodiment 500. The example block diagram of FIG. 5 may be implemented using neuromodulator action policy generator 200 of FIG. 1.

The process may begin by accessing data associated with a specific patient cohort, such as cohort 502. This cohort may consist of a group of patients sharing commonalities, defined by factors such as similar symptom characteristics (e.g., tremor severity), anatomical considerations, or physiological factors, among others. The data related to this cohort may be a compilation of patient information, neuromodulator device settings, and frequently collected data such as patient-reported outcomes, device telemetry data, and more. This data may provide an understanding of each patient's unique characteristics, their specific symptom profiles (like tremors in Parkinson's), and how they respond to different neuromodulation treatment modalities.

Proceeding to block 504, the process may perform neuromodulator data clustering, leading to the generation of distinct neuromodulator settings clusters, such as neuromodulator settings clusters 506, 508, and 510. This step may involve analyzing patterns within the neuromodulator device settings and grouping settings with similar characteristics into distinct clusters, and it may involve utilizing machine learning algorithms. A setting could encompass a wide variety of parameters such as the number of active leads, their specific configuration, pulse amplitude, width, frequency, and/or waveform parameters. These clusters may represent the common neuromodulator settings patterns across the patient cohort, thereby enabling the recommendation of personalized treatment strategies.

In the illustrative embodiment, the clustering process leverages neuromodulator data obtained from patient cohort 502. The data used may incorporate initial neuromodulator device settings, applied at the commencement of the treatment, and any subsequent adjustments made during the course of treatment. This approach may facilitate a dynamic and evolving view of the treatment, thus allowing for the identification of various treatment strategies and how they change over time, an aspect that could be helpful for optimizing patient outcomes over time.

As an example, the neuromodulator setting cluster 506 may represent a subset of neuromodulator device settings typically utilized for patients dealing with specific symptom characteristics, such as tremors associated with Parkinson's. This cluster might be characterized by settings with a specific frequency, pulse width, or unique electrode configuration, which may have been observed to be particularly effective for patients with such symptom profiles. Here, the process (e.g., through machine learning algorithms) may identify patterns such as an increased level of symptom relief (reduced tremors) when a certain frequency is used, or a specific electrode configuration is deployed.

Similarly, neuromodulator setting cluster 508 might denote another unique subset of neuromodulator settings, perhaps involving the use of higher amplitude settings. This setting could be beneficial for patients who have developed a certain level of tolerance to the stimulation over time or for those dealing with more severe symptoms. Observations within this cluster might reveal correlations like higher amplitude settings, in conjunction with specific lead positions, may yield better symptom management outcomes in patients with severe or persistent symptoms.

Finally, neuromodulator setting cluster 510 could represent a cluster of settings that make use of unconventional or advanced waveform parameters. This cluster might be geared towards patients who present complex symptom conditions and have not achieved adequate relief with traditional neuromodulator treatment parameters. These detailed insights gathered from each cluster can be beneficial for tailoring neuromodulation therapy according to the specific needs of the patients. This facilitates a more personalized, effective approach to treatment.

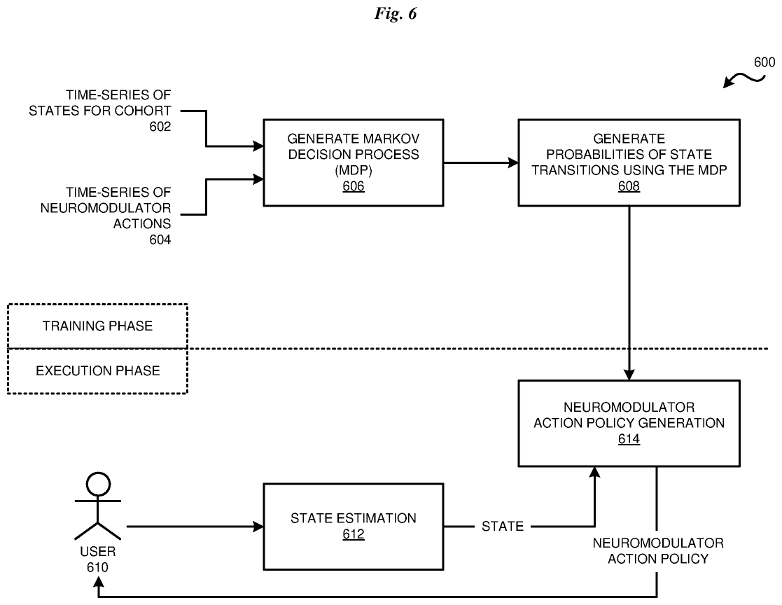

With reference to FIG. 6, this figure depicts a block diagram of an example process for generating a neuromodulator action policy in accordance with an illustrative embodiment 600. The example block diagram of FIG. 6 may be implemented using neuromodulator action policy generator 200 of FIG. 1.

The illustrative embodiment is divided into a training phase and an execution phase. This bifurcation may represent a learning phase as applied to historic patient information and a real-time application of this learned knowledge for serving the needs of the user.

The training phase may represent a learning stage where the system ingests and processes a vast repository of historical data from various patients. This data may include a variety of variables such as patient physiological data, patient-reported symptoms like tremors or bradykinesia, mood changes, treatment responses, among other information. This data may be processed using machine learning algorithms for identifying patterns and trends, segmenting patients into cohorts based on similarities, discovering unique states within these cohorts, and grouping neuromodulator settings, among other functions. The aim of this phase may be to uncover insights from the data that can facilitate the creation of a robust and efficient recommendation system.

In contrast, the execution phase may represent the application of the knowledge gained during the training phase. This process may involve real-time data ingestion and analysis, cohort determination for the patient based on the new data, estimation of the current patient's state, and the application of a trained model to decide upon the optimal neuromodulator action, among other functions. This phase may be an exercise in making real-time, data-driven decisions to provide the best possible therapeutic outcomes for the user.

At the training phase, at block 606, the process may generate and/or use a Markov decision process (MDP). The MDP creation may involve using data on patient states and associated neuromodulator actions to create a dynamic, probabilistic model that can capture the possible transitions between different patient states. This MDP framework may enable a mathematical approach to solving the problem of finding optimal policies. In other words, MDPs may provide a way of modeling the environment in which the patient exists, enabling the system to make decisions based on probable outcomes.

To generate an MDP, the process may integrate data from multiple sources, such as time-series data of states from a cohort 602 and time-series data of neuromodulator actions 604. Time-series data may encapsulate sequential information, offering insights into the progression of patient conditions and the corresponding changes in neuromodulator settings. The time-series of states might reflect changes in health states of patients within the cohort over a certain period. This could include but is not limited to variables such as symptom level, activity levels, sleep quality, and mood fluctuations. For instance, a state could be defined as “intense tremors, low activity, poor sleep,” and the evolution of this state over time may produce the time-series data. The time-series of neuromodulator actions, conversely, might represent the alterations in neuromodulator device configurations over time, showcasing different therapeutic approaches employed to manage patients' symptoms. This data could highlight changes in the frequency, current levels, voltage, and intensity of the neuromodulator device, among other parameters.

The process may use these data sources for constructing the MDP. With the time-series data of patient states, for instance, it might discern the underlying state-transition dynamics in the patient population. With the time-series data of neuromodulator actions, it could identify how these actions interact with patient states and potentially guide state transitions. This relationship between states and actions could form the MDP's foundation, letting the system predict likely future states based on the current state and action, and subsequently, design optimal policies.

At block 608, the process might generate probabilities of state transitions using the MDP, which could depict potential states a patient might undergo and the potential transitions between states based on the actions of the neuromodulator. A transition from one state to another could be sparked by any change in these parameters, which might then be influenced by specific neuromodulator actions. Using the MDP, the system can compute the odds of moving from one state to another given a particular neuromodulator action.

The MDP, and thus the transition probabilities, can be honed through further training to boost the system's precision and efficacy. This supplementary training might hinge on factors like output accuracy (e.g., comparing the system's predictions against tangible outcomes) or user feedback. If a particular action proposed by the system doesn't deliver the expected benefit, user feedback can guide adjustments to the MDP. Through this cyclical process, the system continually learns, advances, and adapts, striving for the most accurate and helpful therapeutic recommendations.

During the execution phase, the process might consistently receive fresh data from user 610. This data stream can encompass real-time physiological data, patient-reported outcomes, and current neuromodulator device configurations, among other details. This inbound data can serve as the input for the execution recommendation process, driving the decision-making for the prime neuromodulator action.

At block 612, the process may undertake state estimation for the user. This step might entail processing the incoming data and pinpointing the user's present state. This estimate could hinge on various factors like patient-reported outcomes, sensor readings, and current device configurations. Machine learning methods might be deployed for precise state estimation, even amidst ambiguity and incomplete details.

At block 614, the process may formulate a neuromodulator action policy anchored on the user's present state. This policy might describe the best setup for the neuromodulator device that's likeliest to assist the user in shifting from their current state to a preferable one, such as one with fewer tremors and enhanced overall health. This procedure might use the transition probabilities and reward information from the earlier trained MDP. As an illustration, the system might opt for neuromodulator actions with the highest probability to shift from the user's current state to a superior one, which could be marked by reduced tremors, improved mood, or better sleep than the user's present state. If a patient is in a state characterized by intense tremors, reduced activity, and unsatisfactory sleep, the policy might suggest augmenting the intensity of the neuromodulator device. This recommendation would stem from the learned understanding that this action frequently results in a state transition towards diminished tremors, heightened activity, and restorative sleep for similar patients in the same cohort. This policy could be adjusted as more data emerges, guaranteeing it remains congruent with the freshest insights on how states and actions intertwine.

The system might showcase the neuromodulator action policy to the user or instantly tweak the neuromodulator's settings after pinpointing the neuromodulator action policy. If the policy determines that upping the voltage of the DBS device would likely lead to a better state for the patient, for instance, this suggestion could be displayed to the patient or their healthcare provider via an intuitive interface, such as an app on a smartphone or a display on the DBS device. They could then implement the recommended changes manually. Additionally or alternatively, the system could possess the capability to independently adjust the DBS device's settings in response to the prevailing patient state, optimizing the patient's therapy without manual interference. This direct alteration might pave the way for a more streamlined and potent response, magnifying the overall treatment's efficacy.

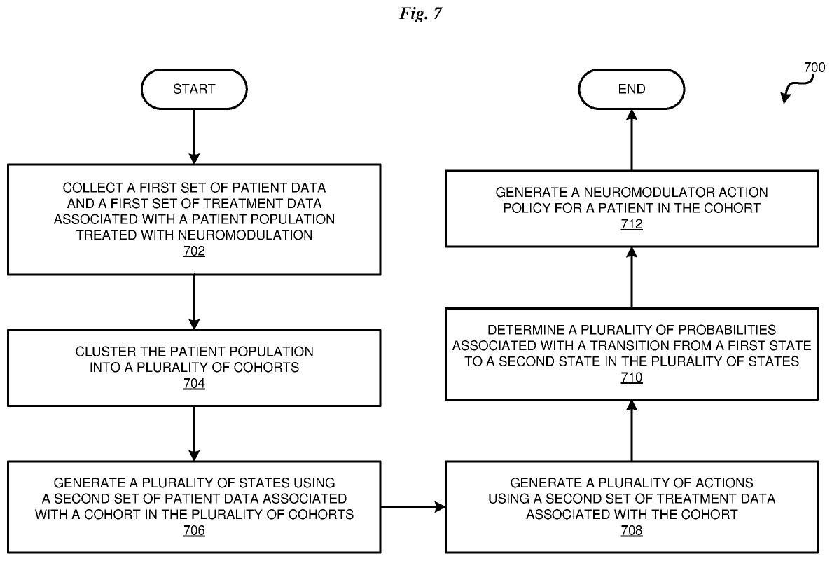

With reference to FIG. 7, this figure depicts a block diagram of an example process for adaptive neuromodulator action policy generation in accordance with an illustrative embodiment 700. The example block diagram of FIG. 7 may be implemented using neuromodulator action policy generator 200 of FIG. 1.

In the illustrative embodiment, at block 702, the process collects, by a data collection module, a first set of patient data and a first set of treatment data associated with a patient population treated with neuromodulation. At block 704, the process clusters, by a clustering module, the patient population into a plurality of cohorts. At block 706, the process generates, by a state discovery module, a plurality of states using a second set of patient data associated with a cohort in the plurality of cohorts. At block 708, the process generates, by an action discovery module, a plurality of actions using a second set of treatment data associated with the cohort. At block 710, the process determines, by a decision-making module based on the plurality of actions, a plurality of probabilities associated with a transition from a first state to a second state in the plurality of states. At block 712, the process generates, by a recommendation module based on the plurality of probabilities, a neuromodulator action policy for a patient in the cohort. It is to be understood that steps may be skipped, modified, or repeated in the illustrative embodiment. Moreover, the order of the blocks shown is not intended to require the blocks to be performed in the order shown, or any particular order.

The following definitions and abbreviations are to be used for the interpretation of the claims and the specification. As used herein, the terms “comprises,” “comprising.” “includes,” “including.” “has.” “having.” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “illustrative” is used herein to mean “serving as an example, instance or illustration.” Any embodiment or design described herein as “illustrative” is not necessarily to be construed as preferred or advantageous over other embodiments or designs. The terms “at least one” and “one or more” are understood to include any integer number greater than or equal to one, i.e., one, two, three, four, etc. The terms “a plurality” are understood to include any integer number greater than or equal to two, i.e., two, three, four, five, etc. The term “connection” can include an indirect “connection” and a direct “connection.”

References in the specification to “one embodiment.” “an embodiment,” “an example embodiment,” etc., indicate that the embodiment described can include a particular feature, structure, or characteristic, but every embodiment may or may not include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The terms “about.” “substantially.” “approximately.” and variations thereof, are intended to include the degree of error associated with measurement of the particular quantity based upon the equipment available at the time of filing the application. For example, “about” can include a range of ±8% or 5%, or 2% of a given value.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

Thus, a computer implemented method, system or apparatus, and computer program product are provided in the illustrative embodiments for managing participation in online communities and other related features, functions, or operations. Where an embodiment or a portion thereof is described with respect to a type of device, the computer implemented method, system or apparatus, the computer program product, or a portion thereof, are adapted or configured for use with a suitable and comparable manifestation of that type of device.

Where an embodiment is described as implemented in an application, the delivery of the application in a Software as a Service (SaaS) model is contemplated within the scope of the illustrative embodiments. In a SaaS model, the capability of the application implementing an embodiment is provided to a user by executing the application in a cloud infrastructure. The user can access the application using a variety of client devices through a thin client interface such as a web browser (e.g., web-based e-mail), or other light-weight client-applications. The user does not manage or control the underlying cloud infrastructure including the network, servers, operating systems, or the storage of the cloud infrastructure. In some cases, the user may not even manage or control the capabilities of the SaaS application. In some other cases, the SaaS implementation of the application may permit a possible exception of limited user-specific application configuration settings.

Embodiments of the present invention may also be delivered as part of a service engagement with a client corporation, nonprofit organization, government entity, internal organizational structure, or the like. Aspects of these embodiments may include configuring a computer system to perform, and deploying software, hardware, and web services that implement, some or all of the methods described herein. Aspects of these embodiments may also include analyzing the client's operations, creating recommendations responsive to the analysis, building systems that implement portions of the recommendations, integrating the systems into existing processes and infrastructure, metering use of the systems, allocating expenses to users of the systems, and billing for use of the systems. Although the above embodiments of present invention each have been described by stating their individual advantages, respectively, present invention is not limited to a particular combination thereof. To the contrary, such embodiments may also be combined in any way and number according to the intended deployment of present invention without losing their beneficial effects.

What is claimed is:

1. A computer-implemented method comprising:
collecting, by a data collection module, a first set of patient data and a first set of treatment data associated with a patient population treated with neuromodulation;
clustering, by a clustering module, the patient population into a plurality of cohorts;
generating, by a state discovery module, a plurality of states using a second set of patient data associated with a cohort in the plurality of cohorts;
generating, by an action discovery module, a plurality of actions using a second set of treatment data associated with the cohort;
determining, by a decision-making module based on the plurality of actions, a plurality of probabilities associated with a transition from a first state to a second state in the plurality of states; and
generating, by a recommendation module based on the plurality of probabilities, a neuromodulator action policy for a patient in the cohort.

2. The method of claim 1, further comprising:
determining a current state of the patient using time-series patient data associated with the patient; and
determining the neuromodulator action policy by selecting one or more actions with a highest probability to transition from the current state to another state.

3. The method of claim 2, further comprising:
determining a current setting of a neuromodulator using time-series treatment data associated with the patient; and
determining the neuromodulator action policy by selecting a sequence of adjustments to the neuromodulator with the highest probability to transition from the current state to another state.

4. The method of claim 1, further comprising:
generating a Markov decision process using time-series patient data and time-series treatment data associated with the cohort; and
determining the plurality of probabilities using the Markov decision process.

5. The method of claim 4, further comprising:
receiving a user feedback on the neuromodulator action policy; and
adjusting the Markov decision process based on the user feedback.

6. The method of claim 1, wherein a cohort represents a group of patients who share a similarity in at least one of a physiological feature, a symptom diagnosis, a neuromodulator device type, and an implant location.

7. The method of claim 1, further comprising:
presenting the neuromodulator action policy to the patient; and
automatically adjusting a neuromodulator setting based on the neuromodulator action policy.

8. The method of claim 1, wherein:
the patient data includes at least one of a symptom level, an activity level, a sleep quality, and a mood level; and
a state in the plurality of states includes a combination of at least one of the symptom level, the activity level, the sleep quality, and the mood level.

9. The method of claim 1, wherein:
the treatment data includes at least one of a neuromodulator frequency, a neuromodulator current, a neuromodulator voltage, and a neuromodulator intensity; and
the neuromodulator action policy includes adjusting at least one of the neuromodulator frequency, the neuromodulator current, the neuromodulator voltage, and the neuromodulator intensity.

10. A computer program product comprising one or more computer readable storage media, and program instructions collectively stored on the one or more computer readable storage media, the program instructions executable by a processor to cause the processor to perform operations comprising:
collecting, by a data collection module, a first set of patient data and a first set of treatment data associated with a patient population treated with neuromodulation;
clustering, by a clustering module, the patient population into a plurality of cohorts;
generating, by a state discovery module, a plurality of states using a second set of patient data associated with a cohort in the plurality of cohorts;
generating, by an action discovery module, a plurality of actions using a second set of treatment data associated with the cohort;
determining, by a decision-making module based on the plurality of actions, a plurality of probabilities associated with a transition from a first state to a second state in the plurality of states; and
generating, by a recommendation module based on the plurality of probabilities, a neuromodulator action policy for a patient in the cohort.

11. The computer program product of claim 10, further comprising:
determining a current state of the patient using time-series patient data associated with the patient; and
determining the neuromodulator action policy by selecting one or more actions with a highest probability to transition from the current state to another state.

12. The computer program product of claim 11, further comprising:
determining a current setting of a neuromodulator using time-series treatment data associated with the patient; and
determining the neuromodulator action policy by selecting a sequence of adjustments to the neuromodulator with the highest probability to transition from the current state to another state.

13. The computer program product of claim 10, further comprising:
generating a Markov decision process using time-series patient data and time-series treatment data associated with the cohort; and
determining the plurality of probabilities using the Markov decision process.

14. The computer program product of claim 13, further comprising:
receiving a user feedback on the neuromodulator action policy; and
adjusting the Markov decision process based on the user feedback.

15. The computer program product of claim 10, wherein a cohort represents a group of patients who share a similarity in at least one of a physiological feature, a symptom diagnosis, a neuromodulator device type, and an implant location.

16. A computer system comprising a processor and one or more computer readable storage media, and program instructions collectively stored on the one or more computer readable storage media, the program instructions executable by the processor to cause the processor to perform operations comprising:
collecting, by a data collection module, a first set of patient data and a first set of treatment data associated with a patient population treated with neuromodulation;
clustering, by a clustering module, the patient population into a plurality of cohorts;
generating, by a state discovery module, a plurality of states using a second set of patient data associated with a cohort in the plurality of cohorts;
generating, by an action discovery module, a plurality of actions using a second set of treatment data associated with the cohort;
determining, by a decision-making module based on the plurality of actions, a plurality of probabilities associated with a transition from a first state to a second state in the plurality of states; and
generating, by a recommendation module based on the plurality of probabilities, a neuromodulator action policy for a patient in the cohort.

17. The computer system of claim 16, further comprising:
determining a current state of the patient using time-series patient data associated with the patient; and
determining the neuromodulator action policy by selecting one or more actions with a highest probability to transition from the current state to another state.

18. The computer system of claim 17, further comprising:
determining a current setting of a neuromodulator using time-series treatment data associated with the patient; and
determining the neuromodulator action policy by selecting a sequence of adjustments to the neuromodulator with the highest probability to transition from the current state to another state.

19. The computer system of claim 16, further comprising:
generating a Markov decision process using time-series patient data and time-series treatment data associated with the cohort; and
determining the plurality of probabilities using the Markov decision process.

20. The computer system of claim 19, further comprising:
receiving a user feedback on the neuromodulator action policy; and
adjusting the Markov decision process based on the user feedback.
